(19) United States
(12) Patent Application Publication
(10) Pub. No.: US 2011/0213204 A1
(43) Pub. Date: Sep. 1, 2011

(54) ENDOSCOPE SYSTEM AND IMAGING DEVICE THEREOF

(76) Inventor: Osamu KURODA, Saitama (JP)

(21) Appl. No.: 12/956,374

(22) Filed: Nov. 30, 2010

(30) Foreign Application Priority Data
Feb. 26, 2010 (JP) 2010-042739

(51) Int. Cl. A61B 1/045

(52) U.S. Cl. 600/109

(57) ABSTRACT

An imaging device for an endoscope includes an image pickup lens, a spectral characteristics variable optical element, and a CCD. The spectral characteristics variable optical element passes only light of a specific wavelength in accordance with the distance between first and second plates, and reflects light of the other wavelengths. The CCD converts the light reflected from the variable optical element into an image signal. In fluorescence endoscopy, a fluorescent labeling agent is administered on an internal body part to be examined. Application of excitation light to the body part causes the fluorescent labeling agent to emit fluorescence. The variable optical element removes the excitation light from the fluorescence by regulation of the distance between the first and second plates.

### BACKGROUND OF THE INVENTION

1. Field of the Invention

The present invention relates to an endoscope system and an imaging device used therein.

2. Description Related to the Prior Art

There is known an endoscope system that takes a fluorescence image of an internal body part to be examined where a fluorescent labeling agent is administered or injected (refer to Japanese Patent Laid-Open Publication No. 2009-291554, for example). The fluorescent labeling agent is selectively bonded to specific living tissue containing a lesion or the like. Only upon application of excitation light having a specific wavelength, the fluorescent labeling agent makes a transition to an excited state by the excitation light, and emits fluorescence having a wavelength different from that of the excitation light. Fluorescence endoscopy using such a fluorescent labeling agent makes it possible to find out the minute lesion including a cancer or a tumor that is difficult to find out in normal visible-light endoscopy.

In taking the fluorescence image, not only the fluorescence from the body part to be examined but also the excitation light reflected from the body part is incident upon an image sensor. The excitation light reflected back becomes noise in production of the fluorescence image, and causes degradation in image quality of the fluorescence image. Thus, according to the Japanese Patent Laid-Open Publication No. 2009-291554, an excitation light cut filter for removing the excitation light is provided in front of an image sensor for the purpose of preventing entry of the excitation light into the image sensor.

The wavelength of the excitation light depends on the type of the fluorescent labeling agent to be used. Thus, if the excitation light cut filter is fixed in an endoscope, as in the case of the Japanese Patent Laid-Open Publication No. 2009-291554, the endoscope is specific only to that fluorescent labeling agent. To solve this problem, it is conceivable that the excitation light cut filter is made detachable to allow manual exchange of the filter for the one specific to the arbitrary wavelength corresponding to the fluorescent labeling agent to be used, or a rotation mechanism is provided to selectively dispose one of the plural filters in an imaging path and allow selection of the filter specific to the arbitrary wavelength by operation of the rotation mechanism.

In recent years, however, the variety of the fluorescent labeling agents is increased. There are even cases where the plural types of fluorescent labeling agents are used in a single inspection. Thus, as for the manual exchange of the filter, an insert section of the endoscope has to be pulled out of a human body and inserted into the body again, whenever using the different type of fluorescent labeling agent. This compromises convenience, and increases a burden on a patient. As for the automatic exchange of the filter by the rotation mechanism, on the other hand, the pullout and re-insertion of the insert section become unnecessary, but provision of the rotation mechanism causes increase in thickness of the insert section. The thick insert section causes degradation in operatability of the endoscope and increase in a burden on the patient.

### SUMMARY OF THE INVENTION

An object of the present invention is to provide an endoscope system that can carry out fluorescence endoscopy with use of plural types of fluorescent labeling agents without reduction in convenience, increase in a burden on a patient, and increase in the diameter of an insert section, and to provide an imaging device used therein.

To achieve the above and other objects of the present invention, an imaging device according to the present invention includes an image pickup lens, a spectral characteristics variable optical element, and an image sensor. The image pickup lens forms image light from light incident from a section to be examined. The spectral characteristics variable optical element passes the image light of a specific wavelength out of the image light incident from the image pickup lens, and reflecting the image light of the other wavelengths. The spectral characteristics variable optical element includes plural plates disposed in parallel with leaving a predetermined distance and a drive section for varying the distance in accordance with the wavelength of the image light to be passed. Each of the plates has a transparent base and a half mirror film attached to the base. The image sensor captures the image light reflected from the spectral characteristics variable optical element, and outputs an image signal corresponding to the image light.

It is preferable that the spectral characteristics variable optical element be disposed inclinedly at a predetermined angle relative to an optical axis of the image pickup lens, so as to reflect the image light incident from the image pickup lens to the image sensor.

The imaging device may further include a beam splitter disposed between the image pickup lens and the spectral characteristics variable optical element, and a quarter wavelength plate disposed between the beam splitter and the spectral characteristics variable optical element. The beam splitter has an inclined polarization separation film. The polarization separation film passes to the spectral characteristics variable optical element one of linearly P-polarized light and linearly S-polarized light out of the image light incident from the image pickup lens, and reflects to the image sensor the other one of the linearly P-polarized light and the linearly S-polarized light that has returned to the polarization separation film by reflection from the spectral characteristics variable optical element. The spectral characteristics variable optical element is disposed orthogonally to an optical axis of the image pickup lens. The quarter wavelength plate converts one of the linearly P-polarized light and the linearly S-polarized light incident from the beam splitter into circularly polarized light, and converts the circularly polarized light incident from the spectral characteristics variable optical element into the other one of the linearly P-polarized light and the linearly S-polarized light.

The imaging device may further include a light absorber for absorbing and attenuating light that has passed through the spectral characteristic variable optical element. Out of the plural plates, the plate including a light exit surface may have the base of the light absorber.

An endoscope system according to the present invention includes the imaging device described above and a controller. The controller controls a drive section so that the wavelength of the image light passing through the spectral characteristics variable optical element coincides with a wavelength of an excitation light.

The endoscope system may further include a fluorescence wavelength input section for indicating a wavelength of fluorescence, an A/D converter for converting the image signal outputted from the image sensor into digital image data, and a spectral image generator for applying spectral estimation processing to the image data. The spectral characteristics variable optical element excludes from the image signal a signal component based on the image light of the same wavelength as the wavelength of the excitation light. The spectral image generator generates by the spectral estimation processing a spectral image corresponding to the wavelength of the fluorescence indicated by the fluorescence wavelength input section.

The endoscope system may further include an excitation light wavelength input section for indicating a wavelength of the excitation light, and a light source for supplying an endoscope with the excitation light of the wavelength indicated by the excitation light wavelength input section. The controller controls the drive section in accordance with the wavelength of the excitation light indicated by the excitation light wavelength input section. The excitation light travels through a light guide routed through the endoscope to be applied to the section to be examined.

According to the present invention, since the distance between the first and second plates is regulated so that the light of the same wavelength as that of the excitation light passes through the spectral characteristics variable optical element, the spectral characteristics variable optical element can remove a component of the excitation light from the image light incident from the section to be examined. Accordingly, in fluorescence endoscopy using a plurality of types of fluorescent labeling agents, it is possible to eliminate the need for pulling out an insertion section whenever excitation light removal filters are exchanged, and hence prevent deterioration of convenience and increase in a burden of a patient. It is also possible to eliminate the need for providing a plurality of types of filters and a filter exchanging mechanism, and hence prevent increase in a diameter of a distal end of the insertion section.

### BRIEF DESCRIPTION OF THE DRAWINGS

For more complete understanding of the present invention, and the advantage thereof, reference is now made to the following descriptions taken in conjunction with the accompanying drawings, in which:

FIG. 1 is a block diagram of a fluorescence endoscope system;

FIG. 2 is an explanatory view showing the structure of an imaging device according to a first embodiment; and

FIG. 3 is an explanatory view showing the structure of the imaging device according to a second embodiment.

### DESCRIPTION OF THE PREFERRED EMBODIMENTS

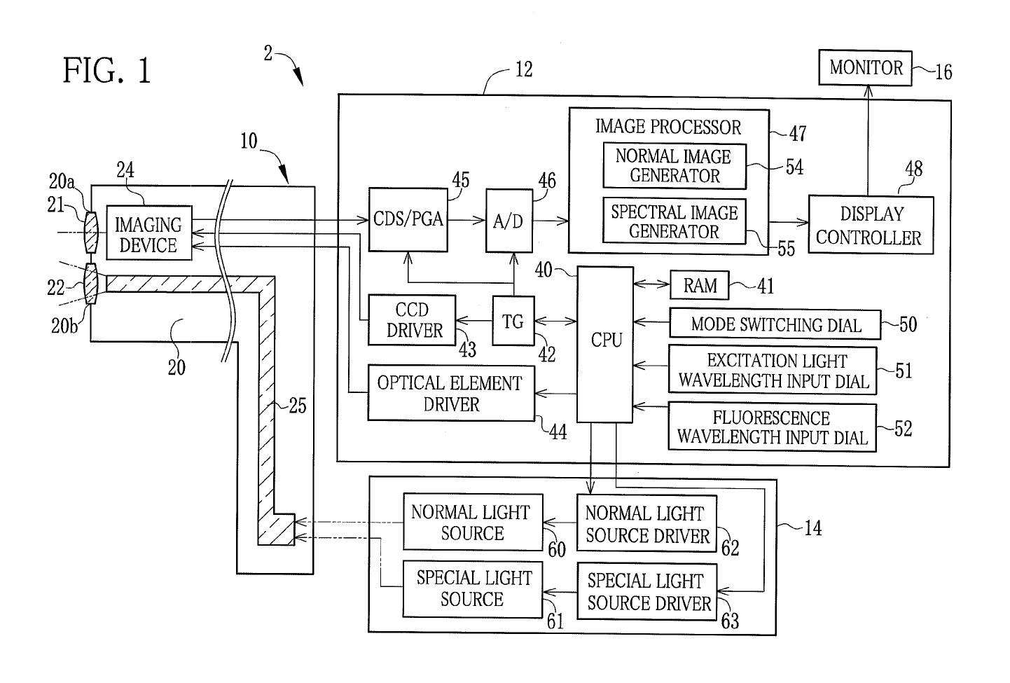

As shown in FIG. 1, a fluorescence endoscope system 2 is constituted of an electronic endoscope 10 for imaging the inside of a patient's body, a processing device 12 for producing an endoscope image, a light source device 14, and a monitor 16 for displaying the endoscope image. The light source device 14 selectively supplies to the electronic endoscope 10 one of white light (normal light) for lighting the inside of the body and excitation light for exciting a fluorescent labeling agent administered on or injected into an internal body part.

This endoscope system 2 has a normal imaging mode and a fluorescence imaging mode. In the normal imaging mode, the normal light is supplied to the electronic endoscope 10 to image the inside of the body part with visible light. In the fluorescence imaging mode, the excitation light is supplied to the electronic endoscope 10 so as to image with fluorescence a tumor or the like to which the administered or injected fluorescent labeling agent is selectively bonded.

In an inspection, a doctor puts the endoscope system 2 into the normal imaging mode to observe the inside of the body illuminated with the white light. If a part suspected of being a lesion is found out, the fluorescent labeling agent is administered on or injected into the part. Then, the endoscope system 2 is switched to the fluorescence imaging mode to take a fluorescence image. Such fluorescence endoscopy using the fluorescent labeling agent facilitates identification of the minute lesion that is difficult to find out with the normal visible light.

The electronic endoscope 10 has a slender and tubular insert section 20 to be inserted into the patient's body. The electronic endoscope 10 has a universal cord (not-illustrated), and detachably connected to the processing device 12 and the light source device 14 through a connector provided at a distal end of the universal cord.

In a distal end of the insert section 20, there are provided an image capturing window 21 for taking in image light from the body part to be examined, and a lighting window 22 for emitting the normal light or the excitation light supplied by the light source device 14. The windows 21 and 22 are fitted into openings 20a and 20b formed in the distal end of the insert section 20 so as to close the openings 20a and 20b, respectively. Window fittings are lenses or plates made of optical glass or optical plastic. Thus, the image light from the internal body part is incident upon the insertion section 20 through the image capturing window 21, and the normal or excitation light from the light source device 14 is emitted through the lighting window 22.

The electronic endoscope 10 contains an imaging device 24 and a light guide 25. The imaging device 24 is disposed so as to face the image capturing window 21, and applies photoelectric conversion to the image light incident through the image capture window 21. The light guide 25 is made of a flexible optical fiber, and guides the normal or excitation light supplied from the light source device 14 to the lighting window 22.

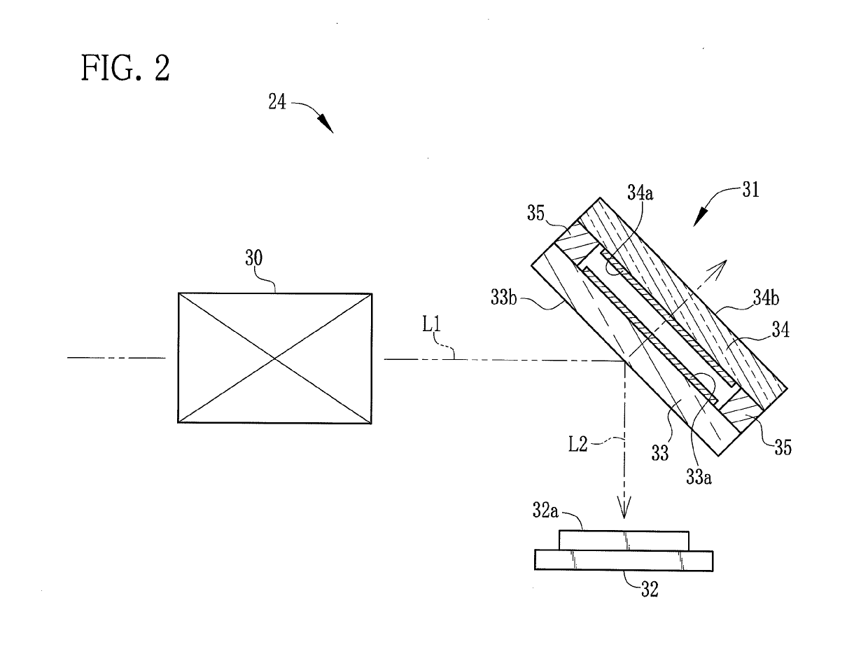

As shown in FIG. 2, the imaging device 24 is constituted of an image pickup lens 30, a spectral characteristics variable optical element 31, and a CCD (image sensor) 32. The image light incident from the image capturing window 21 passes through the image pickup lens 30 and the variable optical element 31, and forms an image on the CCD 32. The variable optical element 31 is approximately in the shape of a plate. The variable optical element 31 is constituted of a first plate 33 and a second plate 34 that are disposed oppositely to each other with leaving a parallel gap, and an actuator (drive section) 35 disposed between the first and second plates 33 and 34. The actuator 35 stretches or shrinks in response to input of a drive signal so as to vary the distance between the plates 33 and 34.

Each plate 33, 34 is constituted of a optical glass or optical plastic base and a half mirror film 33a, 34a attached to the base. This variable optical element 31 is a so-called Fabry-Perot etalon, which allows only light of a specific wavelength to pass therethrough in accordance with the distance between the plates 33 and 34, though reflects light of the other wavelengths. Thus, operation of the actuator 35 varies the distance between the plates 33 and 34, and hence chooses the wavelength of the light to be passed through.

The base of the first plate 33 is made of a colorless transparent material, and has high transmittance. On the other hand, the base of the second plate 34 is a so-called absorption type ND filter in which a light absorbing material is fused with a transparent material, and absorbs and attenuates incident light. The base of the second plate 34 preferably has as high optical density as possible in a wavelength region from ultraviolet to infrared.

A surface 33b of the first plate 33 is a light entrance surface of the variable optical element 31, and a surface 34b of the second plate 34 is a light exit surface thereof. The variable optical element 31 is inclined at approximately 45° relative to an optical axis L1 of the image pickup lens 30. Consequently, the image light that has passed through the image pickup lens 30 is incident upon the variable optical element 31. Out of the image light, the light of the specific wavelength corresponding to the distance between the plates 33 and 34 passes through the variable optical element 31, and the light of the other wavelengths is reflected at an angle of approximately 90°. At this time, the light having passed through the variable optical element 31 is absorbed and attenuated by the second plate 34.

The wavelength of the light passing through the variable optical element 31 depends on the mode of the endoscope system 2. In the fluorescence endoscopy, the excitation light is applied to the internal body part to be examined. The excitation light reflected from the body part is incident through the image capturing window 21, and causes degradation in image quality as noise.

Thus, in the fluorescence imaging mode, the distance between the plates 33 and 34 is set so that the excitation light passes through the variable optical element 31. Therefore, when the image light that has passed through the image pickup lens 30 is incident upon the variable optical element 31, a component of the excitation light out of the image light passes through the first plate 33, and light of the other wavelengths containing a component of the fluorescence is reflected from the first plate 33 to enter the CCD 32. As described above, in the fluorescence endoscopy, the variable optical element 31 is used as a filter to remove the component of the excitation light contained in the image light.

The excitation light that has passed through the variable optical element 31 is absorbed by the second plate 34, as described above. The second plate 34 prevents that the excitation light that has passed through the variable optical element 31 is reflected from another element and travels to the image sensor 32, or travels in the variable optical element 31 in an opposite direction from a side of the second plate 34 and passes through the first plate 33.

When the endoscope system 2 is put into the normal imaging mode, on the other hand, the distance between the plates 33 and 34 is set so that light having wavelengths outside the visible region, including light in the ultraviolet region and the infrared region passes through the variable optical element 31. Thus, in the normal imaging mode, the visible image light, which is necessary for normal endoscopy, is reflected and incident upon the CCD 32.

The CCD 32 is disposed so that a light receiving surface 32a is approximately orthogonal to an optical axis L2. Thus, the light reflected from the variable optical element 31 is incident upon the light receiving surface 32a of the CCD 32. In other words, the visible image light is incident upon the light receiving surface 32a when the endoscope system 2 is put into the normal imaging mode, while the light after filtering out the excitation light is incident as the image light upon the light receiving surface 32a when the endoscope system 2 is put into the fluorescence imaging mode. The CCD 32 applies the photoelectric conversion to the image light reflected from the variable optical element 31, and outputs an image signal. The CCD 32 is a widely known color CCD having a color mosaic filter attached to the light receiving surface 32a, and outputs the color image signal.

As shown in FIG. 1, the processing device 12 includes a CPU 40, a RAM 41, a timing generator (TG) 42, a CCD driver 43, an optical element driver 44, a correlated double sampling/programmable gain amplifier (CDS/PGA) 45, an A/D converter (A/D) 46, an image processor 47, and a display controller 48.

The RAM 41 stores various types of programs and data used in control of the processing device 12. The CPU 40 reads out the programs from the RAM 41, and successively executes the programs to control individual parts of the processing device 12 altogether.

To the CPU 40, a mode switching dial 50 for switching setting of the imaging mode, an excitation light wavelength input dial 51, and a fluorescence wavelength input dial 52 are connected. The excitation light wavelength input dial 51 is used for selectively indicating the wavelength of the excitation light depending on the fluorescent labeling agent to be used. The fluorescence wavelength input dial 52 is used for indicating the wavelength of the fluorescence emitted from the body part in accordance with the type of the administered or injected fluorescent labeling agent. The mode switching dial 50 and the wavelength input dials 51 and 52 may be provided on the light source device 14, a handle of the electronic endoscope 10, or the like, instead of the processing device 12.

The TG 42 inputs a timing signal (clock pulses) to the CCD driver 43 under control of the CPU 40. The CCD driver 43 inputs a drive signal to the CCD 32 based on the inputted timing signal, in order to control readout timing of electric charges accumulated in the CCD 32, a shutter speed of an electronic shutter of the CCD 32, and the like.

Under the control of the CPU 40, the optical element driver 44 inputs the drive signal to the actuator 35 of the variable optical element 31 to vary the distance between the plates 33 and 34. In the normal imaging mode, the CPU 40 controls the optical element driver 44 so that the light of the wavelengths outside the visible region, including the light in the ultraviolet region and the infrared region passes through the variable optical element 31. In the fluorescence imaging mode, on the other hand, the CPU 40 controls the optical element driver 44 so that the light having the same wavelength as that of the excitation light indicated by the excitation light wavelength input dial 51 passes through the variable optical element 31.

The CDS/PGA 45 applies noise removal and amplification to the image signal, which is outputted from the CCD 32 under the control of the CCD driver 43, and outputs the processed image signal to the A/D 46. The A/D 46 converts the analog image signal outputted from the CDS/PGA 45 into digital image data, and outputs the digital image data to the image processor 47.

The image processor 47 includes a normal image generator 54 and a spectral image generator 55. The image processor 47 actuates the normal image generator 54 in the normal imaging mode, and actuates the spectral image generator 55 in the fluorescence imaging mode. The normal image generator 54 applies various types of image processing to the image data digitized by the A/D 46 to generate a normal image corresponding to the visible image light. Then the normal image generator 54 outputs the normal image to the display controller 48.

The spectral image generator 55 applies to the image data digitized by the A/D 46 linear approximation by dimensional compression using principal component analysis and spectral estimation processing by Wiener estimation or the like. In the spectral estimation processing, a spectral reflectance of the body part in the visible wavelength region (400 to 700 nm) is estimated from the image data on a pixel of the CCD 32 basis. To be more specific, matrix coefficients are obtained by experiments at a regular interval (for example, 5 nm) of a wavelength, and are stored on a memory. The matrix coefficient that corresponds to the wavelength indicated by the fluorescence wavelength input dial 52 is read out, and the three-color image data is subjected to matrix calculation to produce spectral image data of the designated wavelength. The image data is sent to the display controller 48. If three spectral images are produced with designation of three wavelengths, and are assigned to B, G, and R, respectively, a color composite image is obtained.

The display controller 48 converts the normal image or the spectral image outputted from the image processor 47 into an ATSC-format video signal (component signal, composite signal, and the like) compliant to the monitor 16, and outputs the video signal to the monitor 16. Thus, the normal image corresponding to the wavelength region of the visible light, or the spectral image corresponding to the wavelength region of the fluorescence from the body part to be examined is displayed on the monitor 16 as the endoscope image, which images the inside of the patient's body.

The light source device 14 includes a normal light source 60 for emitting the normal light, a special light source 61 for emitting the excitation light, a normal light source driver 62 for turning on or off the normal light source 60, and a special light source driver 63 for turning on or off the special light source 61. The normal light source 60 emits as the normal light the white light having a relatively flat wavelength characteristic throughout the whole visible region. For example, a xenon lamp is used in the normal light source 60. The special light source 61 uses a wavelength-variable laser, which can arbitrarily change the wavelength of laser light in a range from the ultraviolet to the infrared. The light emitted from each light source 60, 61 travels through a light path (not illustrated), and enters into the light guide 25 of the electronic endoscope 10 connected to the light source device 14. Thus, the electronic endoscope 10 is supplied with the normal light or the excitation light.

The light source drivers 62 and 63 are electrically connected to the CPU 40 of the processing device 12, and turn on or off the light sources 60 and 61, respectively, in response to a control signal from the CPU 40. In the normal imaging mode, the CPU 40 sends the control signal to the normal light source driver 62 to turn on the normal light source 60.

In the fluorescence imaging mode, the CPU 40 sends the control signal to the special light source driver 63 to turn on the special light source 61. At this time, the control signal includes information of the wavelength indicated by the excitation light wavelength input dial 51. Upon reception of the control signal, the special light source driver 63 drives the special light source 61 to emit the excitation light of the wavelength indicated by the excitation light wavelength input dial 51.

Next, operation of the endoscope system 2 having above structure will be described. To make the inspection with use of the endoscope system 2, each part of the endoscope system 2 shown in FIG. 1 is first set up. After the setup, the mode switching dial 50 is operated to put the endoscope system 2 into the normal imaging mode. After that, each part is powered on to actuate the endoscope system 2 in the normal imaging mode.

When the endoscope system 2 is actuated in the normal imaging mode, the CPU 40 of the processing device 12 sends the control signal to the normal light source driver 62 to turn on the normal light source 60, and controls the TG 42 to drive the CCD 32 of the imaging device 24. At the same time, the CPU 40 controls the optical element driver 44 to drive the actuator 35 of the variable optical element 31. Thus, the actuator 35 varies the distance between the first and second plates 33 and 34 so that the light other than the visible light passes through the variable optical element 31. Consequently, the visible image light reflected from the variable optical element 31 is incident upon the CCD 32, and the CCD 32 outputs the image signal corresponding to the image light.

The image signal from the CCD 32 is inputted to the CDS/PGA 45 of the processing device 12. The CDS/PGA 45 applies the noise removal and amplification to the inputted image signal, and outputs the processed image signal to the A/D 46. The A/D 46 digitizes the inputted image signal, and outputs the digital image data to the image processor 47. Upon input of the image data, the image processor 47 actuates the normal image generator 54. The normal image generator 54 generates the normal image from the image data, and outputs the normal image to the display controller 48. The display controller 48 converts the inputted normal image into the video signal, and outputs the video signal to the monitor 16. Thus, the normal image (color image) corresponding to the visible wavelength region is displayed on the monitor 16 as the endoscope image.

After the normal light is applied from the normal light source 60 through the lighting window 22, and the normal image is displayed on the monitor 16, the insert section 20 is inserted into the patient's body to start the inspection. If the part suspected of being the lesion is found out inside the patient's body, the endoscope system 2 is switched from the normal imaging mode to the fluorescence imaging mode.

Before switching to the fluorescence imaging mode, a nozzle is inserted into a forceps channel to administer on the part suspected of being the lesion the fluorescent labeling agent that meets an application of the inspection. After the administration of the fluorescent labeling agent, the excitation light wavelength input dial 51 is operated to input the wavelength of the excitation light of the fluorescent labeling agent, and the fluorescence wavelength input dial 52 is operated to input the wavelength of the fluorescence. Then, the endoscope system 2 is switched from the normal imaging mode to the fluorescence imaging mode by the operation of the mode switching dial 50. The nozzle is pulled out of the forceps channel after the administration of the fluorescent labeling agent.

In the fluorescence imaging mode, the CPU 40 of the processing device 12 stops sending the control signal to the normal light source driver 62 to turn off the normal light source 60, and then sends the control signal to the special light source driver 63 to turn on the special light source 61. Thus, the special light source 61 emits the light of the wavelength indicated by the excitation light wavelength input dial 51 as the excitation light.

Simultaneously, the CPU 40 controls the optical element driver 44 to drive the actuator 35 of the variable optical element 31. Thus, the actuator 35 varies the distance between the first and second plates 33 and 34 so that the excitation light having the wavelength that is indicated by the excitation light wavelength input dial 51 passes through the variable optical element 31. Consequently, the light excluding the excitation light is reflected from the variable optical element 31, and captured by the CCD 32. The CCD 32 outputs the image signal corresponding to the light.

At this time, the excitation light that has passed through the variable optical element 31 is absorbed and attenuated by the second plate 34. Therefore, the second plate 34 prevents that the excitation light that has passed through the variable optical element 31 is reflected from the other element and travels in the variable optical element 31 in the opposite direction from the side of the second plate 34 and passes through the first plate 33. As a result, it is possible to appropriately prevent degradation in the image quality of the endoscope image due to the excitation light.

The image signal outputted form the CCD 32 is subjected to noise removal processing, amplification processing, and A/D conversion processing, and then is inputted to the image processor 47, as in the case of the normal imaging mode. Upon input of the image data, the image processor 47 actuates the spectral image generator 55. The spectral image generator 55 generates the spectral image in which only part corresponding to the fluorescence of the wavelength region indicated by the fluorescence wavelength input dial 52 is extracted, and sends the spectral image to the display controller 48.

The display controller 48 converts the inputted spectral image into the video signal, and outputs the video signal to the monitor 16. Thus, the spectral image that corresponds to the fluorescence emitted from the body part to be examined is displayed as the endoscope image on the monitor 16.

The doctor observes the spectral image displayed on the monitor, and inspects emission of the fluorescence from the body part where the fluorescent labeling agent is administered or injected in detail in order to identify the presence or absence of the lesion. Another fluorescence inspection may be carried out if necessary, after administration or injection of another type of fluorescent labeling agent.

In carrying out the additional fluorescence inspection with use of the other fluorescent labeling agent, as in the case of the first inspection, the fluorescent labeling agent is administered on the body part to be examined with use of the nozzle and the like. Then, the wavelength input dials 51 and 52 are operated to re-input the wavelengths of the excitation light and the fluorescence in accordance with the fluorescent labeling agent.

Upon operation of the excitation light wavelength input dial 51, the CPU 40 modifies the control signal inputted to the special light source driver 63 in response to the operation, so that the special light source 61 emits the excitation light of the newly set wavelength. At the same time, the CPU 40 makes the optical element driver 44 drive the actuator 35 of the variable optical element 31. Thus, the actuator 35 varies the distance between the first and second plates 33 and 34 so that the excitation light of the newly set wavelength passes through the variable optical element 31. Therefore, the excitation light corresponding to the newly administered fluorescent labeling agent is emitted from the lighting window 22, and the spectral image by the fluorescent labeling agent is displayed on the monitor 16.

As described above, in this embodiment, the fluorescence endoscopy using the plural types of fluorescent labeling agents can be carried out only with operation of the excitation light wavelength input dial 51 and the fluorescence wavelength input dial 52, without pulling out the insertion section 20 and exchanging excitation light removal filters. As a result, the endoscope system 2 according to this embodiment does not cause impairment of convenience and increase in a burden of the patient.

In the first embodiment, the variable optical element 31 removes the component of the excitation light out of the image light from the body part to be examined. The variable optical element 31 can remove the excitation light of an arbitrary wavelength only by varying the distance between the first and second plates 33 and 34, and eliminates the need for providing a plurality of filters and a filter exchanging mechanism in the insert section 20. This eliminates the need for increase in the diameter of the insert section 20.

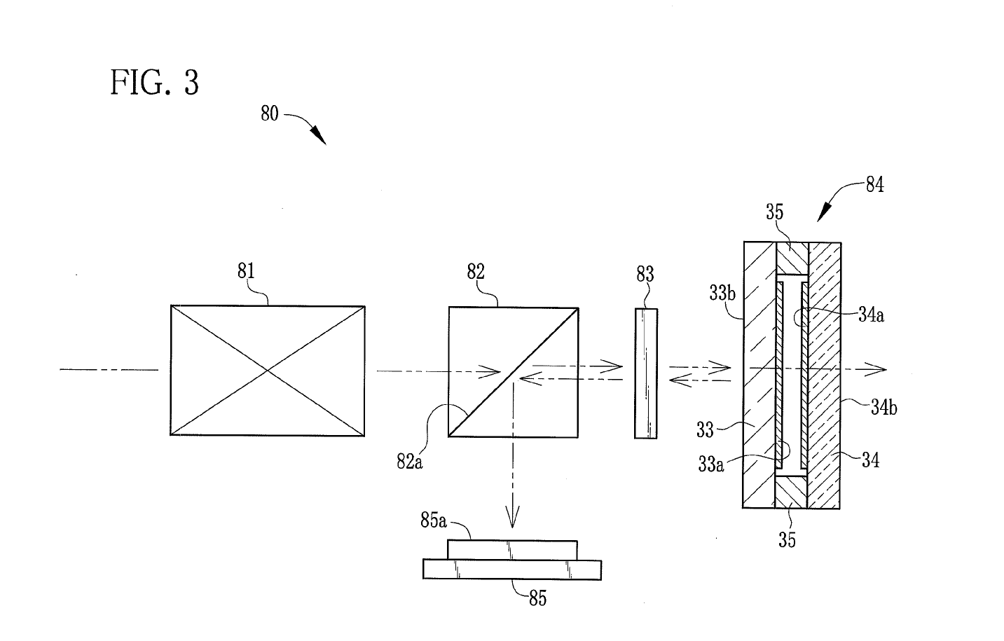

Next, a second embodiment will be described. In the second embodiment, the same reference numbers as those of the first embodiment indicate components having the same function and structure as those of the first embodiment, and detailed description thereof will be omitted. As shown in FIG. 3, an imaging device 80 according to the second embodiment is constituted of an image pickup lens 81, polarization beam splitter 82, a quarter wavelength plate 83, a spectral characteristics variable optical element 84, and a CCD 85.

As in the case of the above first embodiment, the image pickup lens 81 forms an image on the polarization beam splitter 82 from the image light incident from the image capturing window 21. The polarization beam splitter 82 has a polarization separation film 82a having the function of passing linearly P-polarized light and reflecting linearly S-polarized light. The quarter wavelength plate 83 converts the P-polarized image light that has passed through the polarization beam splitter 82 into circularly polarized image light. The circularly polarized image light is incident upon the variable optical element 84.

The variable optical element 84, as with the variable optical element 31 of the first embodiment, is constituted of the first and second plates 33 and 34 and the actuator 35. The distance between the first and second plates 33 and 34 is set so that the light other than the visible light passes through in the normal imaging mode, while the excitation light passes through in the fluorescence imaging mode, as in the case of the first embodiment.

The variable optical element 84 is disposed in such a manner that the surface 33b of the first plate 33, being the light entrance surface, is approximately orthogonal to an optical axis of the quarter wavelength plate 83. Thus, the image light reflected from the variable optical element 84 is incident again upon the quarter wavelength plate 83. The quarter wavelength plate 83 converts the circular polarized image light incident from the variable optical element 84 into linearly polarized image light. The linearly polarized image light is incident upon the polarization beam splitter 82. The reflection by the variable optical element 84 reverses the direction of rotation of the image light. In other words, the rotation direction of the circular polarized image light reflected from the variable optical element 84 is opposite from that incident thereon. Accordingly, the image light that is incident from the variable optical element 84 upon the quarter wavelength plate 83 is converted into linearly polarized image light rotated at 90° relative to the image light incident from the polarization beam splitter 82, that is, the linear S-polarized light.

The polarization beam splitter 82 reflects the S-polarized image light incident from the quarter wavelength plate 83 by the polarization separation film 82a, and leads the S-polarized image light into a light receiving surface 85a of the CCD 85. Thus, the visible image light is incident upon the light receiving surface 85a in the normal imaging mode, while the light excluding the light of the same wavelength as that of the excitation light is incident thereon in the fluorescence imaging mode. The CCD 85 captures the image light incident upon the light receiving surface 85a, and outputs the image signal corresponding to the image light.

Since the imaging device 80 having the above structure can remove the component of the excitation light from the image light from the body part to be examined, the same effect as that of the first embodiment is obtained.

In the above embodiments, the image capturing window is excluded from the imaging device. However, a lens may be fitted into a window frame of the image capturing window, and the imaging device may include the image capturing window.

In the above embodiments, the variable optical element has two plates, but may have three or more plates. The base of the second plate is a light absorber, but may be transparent. In this case, the ND filter is glued to a rear surface of the second plate.

In the above embodiments, the CCD is used as the image sensor, but another type of well-known image sensor including a CMOS image sensor is available instead. In the above embodiment, the wavelength input dials are designated as an excitation light wavelength input section and a fluorescence wavelength input section, but an input device into which a wavelength is inputted in the form of a numeric value may be used instead.

In the above embodiment, the image processor includes the spectral image generator to generate the spectral image from the image data corresponding to the image light that excludes the light of the same wavelength as that of the excitation light. However, the fluorescence image may be directly displayed. Otherwise, the spectral image of an arbitrary wavelength may be generated from the visible light image.

In the above embodiments, the present invention is applied to the endoscope system having the processing device and the light source device, but may be applied to an endoscope system into which the processing device and the light source device are integrated. In the above embodiments, the excitation light is applied from the electronic endoscope. However, a light guide may be inserted into the forceps channel of the electric endoscope, and the excitation light traveling through the light guide may be applied instead.

The above embodiments take a medical endoscope as an example, but the present invention is also applicable to any type of endoscopes including an industrial endoscope for imaging the inside of a machine, a narrow pipe, or the like.

Although the present invention has been fully described by the way of the preferred embodiment thereof with reference to the accompanying drawings, various changes and modifications will be apparent to those having skill in this field. Therefore, unless otherwise these changes and modifications depart from the scope of the present invention, they should be construed as included therein.

What is claimed is:

1. An imaging device for an endoscope comprising:
an image pickup lens for forming image light from light incident from a section to be examined;
a spectral characteristics variable optical element for passing the image light of a specific wavelength out of the image light incident from the image pickup lens, and reflecting the image light of the other wavelengths, the spectral characteristics variable optical element including plural plates disposed in parallel with leaving a predetermined distance and a drive section for varying the distance in accordance with the wavelength of the image light to be passed, each of the plates having a transparent base and a half mirror film attached to the base; and
an image sensor for capturing the image light reflected from the spectral characteristics variable optical element, and outputting an image signal corresponding to the image light.

2. The imaging device according to claim 1, wherein the spectral characteristics variable optical element is disposed inclinedly at a predetermined angle relative to an optical axis of the image pickup lens, so as to reflect the image light incident from the image pickup lens to the image sensor.

3. The imaging device according to claim 1, further comprising:
a beam splitter disposed between the image pickup lens and the spectral characteristics variable optical element, the beam splitter having an inclined polarization separation film for passing to the spectral characteristics variable optical element one of linearly P-polarized light and linearly S-polarized light out of the image light incident from the image pickup lens, and reflecting to the image sensor the other one of the linearly P-polarized light and the linearly S-polarized light having returned to the polarization separation film by reflection from the spectral characteristics variable optical element, the spectral characteristics variable optical element being disposed orthogonally to an optical axis of the image pickup lens; and
a quarter wavelength plate disposed between the beam splitter and the spectral characteristics variable optical element, the quarter wavelength plate for converting one of the linearly P-polarized light and the linearly S-polarized light incident from the beam splitter into circularly polarized light, and converting the circularly polarized light incident from the spectral characteristics variable optical element into the other one of the linearly P-polarized light and the linearly S-polarized light.

4. The imaging device according to claim 1, further comprising:
a light absorber for absorbing and attenuating light having passed through the spectral characteristic variable optical element.

5. The imaging device according to claim 4, wherein out of the plural plates, the plate including a light exit surface has the base of the light absorber.

6. An endoscope system including an endoscope for applying excitation light for exciting a fluorescent substance to a section to be examined, and capturing fluorescence emitted by application of the excitation light from the section to be examined as image light, the endoscope system comprising:
(A) an imaging device for the endoscope including:
an image pickup lens for forming the image light from light incident from the section to be examined;
a spectral characteristics variable optical element for passing the image light of a specific wavelength out of the image light incident from the image pickup lens, and reflecting the image light of the other wavelengths, the spectral characteristics variable optical element including plural plates disposed in parallel with leaving a predetermined distance and a drive section for varying the distance in accordance with the wavelength of the image light to be passed, each of the plates having a transparent base and a half mirror film attached to the base; and
an image sensor for capturing the image light reflected from the spectral characteristics variable optical element, and outputting an image signal corresponding to the image light; and
(B) a controller for controlling the drive section so that the wavelength of the image light passing through the spectral characteristics variable optical element coincides with a wavelength of the excitation light.

7. The endoscope system according to claim 6, further comprising:
(C) a fluorescence wavelength input section for indicating a wavelength of the fluorescence;
(D) an A/D converter for converting the image signal outputted from the image sensor into digital image data, the spectral characteristics variable optical element excluding from the image signal a signal component based on the image light of the same wavelength as the wavelength of the excitation light; and
(E) a spectral image generator for applying spectral estimation processing to the image data, and generating by the spectral estimation processing a spectral image corresponding to the wavelength of the fluorescence indicated by the fluorescence wavelength input section.

8. The endoscope system according to claim 7, further comprising:
(F) an excitation light wavelength input section for indicating a wavelength of the excitation light, the controller controlling the drive section in accordance with the wavelength of the excitation light indicated by the excitation light wavelength input section; and
(G) a light source for supplying the endoscope with the excitation light of the wavelength indicated by the excitation light wavelength input section, the excitation light traveling through a light guide routed through the endoscope, and being applied to the section to be examined.
